(19) United States
(12) Patent Application Publication
(10) Pub. No.: US 2010/0145359 A1
(43) Pub. Date: Jun. 10, 2010

(54) CALLUS RASP

(75) Inventor: Bernhard S. Keller, Burgdorf (CH)

Correspondence Address: GIFFORD, KRASS, SPRINKLE,ANDERSON & CITKOWSKI, P.C, PO BOX 7021, TROY, MI, 48007-7021, US

(73) Assignee: Simon Keller AG

(21) Appl. No.: 12/398,665

(22) Filed: Mar. 5, 2009

(30) Foreign Application Priority Data
Dec. 8, 2008 (DE) 202008016249.1

(51) Int. Cl. A61B 17/50; B32B 37/02

(52) U.S. Cl. 606/131; 156/249

(57) ABSTRACT

A callus rasp is provided that includes a rasp body and an abrasive surface. The rasp body has a handle portion and a blade portion. The rasp body is formed of a nonporous metal, glass or glazed ceramic and amenable to sterilization. The abrasive surface has particulate grit exposed therefrom and with an adhesive intermediate between the abrasive surface and the blade portion, the abrasive surface selectively secured to the blade portion for use as a callus rasp. The abrasive surface is pre-adhesed with the adhesive and a peelable backing layer over the adhesive together form a foil. The foil and rasp body are sterilized before joinder. This callus rasp is amenable to removal of the abrasive surface and sterilization of the rasp body for reuse with a new sterile abrasive surface.

### CROSS-REFERENCE TO RELATED APPLICATIONS

This application claims priority benefit of German Petty Patent Application No. 202008016249.1 filed Dec. 8, 2008, the contents of which are hereby incorporated by reference.

### FIELD OF THE INVENTION

The present invention in general relates to a dermal callus abrading rasp and in particular to a dermal callus abrading rasp having a reusable handle amenable to autoclave sterilization.

### BACKGROUND OF THE INVENTION

Dermal callus removal is a routine procedure performed by podiatrists. Conventional wisdom for many years was that removal of extraneous stratum corneum layers without damage to the underlying living dermal cells or capillary bed posed no health concerns. This approach led to the routine practice of the reuse of professional quality rasps among various individuals with only nominal soap and water cleaning intermediate between rasp uses. There is now a growing appreciation that stratum corneum abrasion without visible damage to the underlying stratum granulosum nonetheless can inoculate an individual with potentially harmful microbes and potentially elicit an immune response to the debris from a previous patient transferred by a reusable callus rasp.

Thus, there exists a need for a callus rasp that is amenable to sterilization while retaining the tool feel of a professional quality callus rasp.

### SUMMARY OF THE INVENTION

A callus rasp is provided that includes a rasp body and an abrasive surface. The rasp body has a handle portion and a blade portion. The rasp body is formed of a nonporous metal, glass or glazed ceramic and amenable to sterilization. The abrasive surface has particulate grit exposed therefrom and with an adhesive intermediate between the abrasive surface and the blade portion, the abrasive surface is selectively secured to the blade portion for use as a callus rasp. The abrasive surface is pre-adhesed with the adhesive and a peelable backing layer over the adhesive together form a foil and packaged for single use in a package amenable to sterilization. The foil is optionally precut to complement a face of the blade portion or alternatively the foil is custom cut for attachment to the blade portion. A second abrasive surface is optionally also applied to the blade portion with the same grit or a different grit relative to the abrasive surface. This callus rasp is amenable to removal of the abrasive surface and sterilization of the rasp body for reuse with a new abrasive surface.

### BRIEF DESCRIPTION OF THE DRAWINGS

FIG. 1 is a perspective exploded view of an inventive callus rasp; and

FIG. 2 is a perspective view of the inventive callus rasp in assembled form.

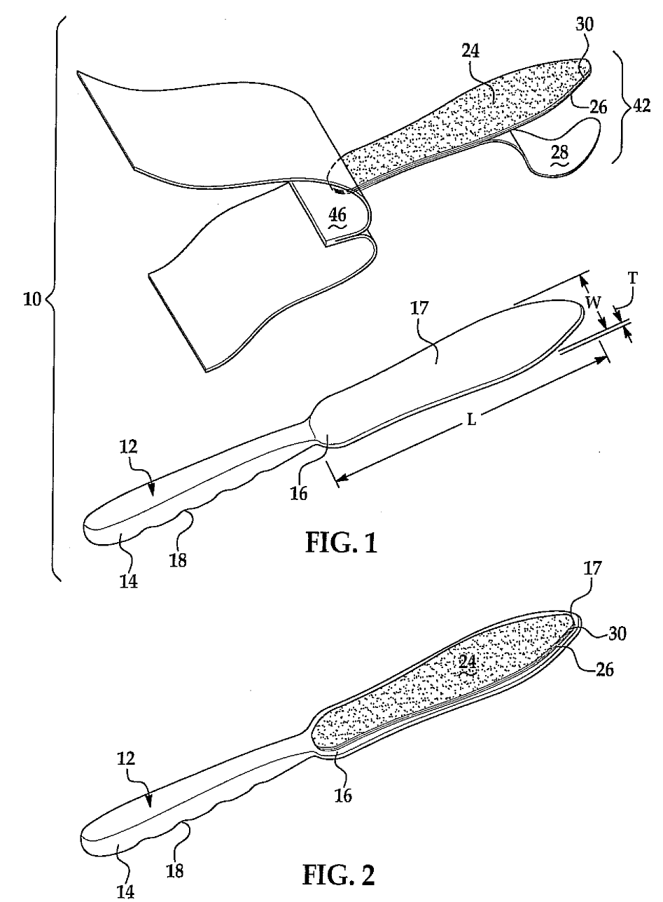

### DETAILED DESCRIPTION OF THE PREFERRED EMBODIMENTS

The present invention has utility as a hygienic callus rasp. A unitary rasp body has a handle portion and a blade portion. The rasp body is formed of a metal or composite material amenable to sterilization between uses. A single use abrasive sheet is secured to the blade portion of the rasp body for usage and then the abrasive sheet is removed to allow for sterilization of the reusable rasp body. Through the use of an inventive callus rasp in this way, callus abrasion-induced infections are precluded.

Referring now to the figures, an inventive callus rasp is shown generally at 10. The rasp 10 has a rasp body 12 having a handle portion 14 and a blade portion 16. The handle portion 14 has a size and shape adapted to fit within the palm of an adult human hand and optionally has knurling, ergonomic recesses, or a combination thereof that define a portion of the surface 18 of the handle portion 14. The blade portion 16 has a length L that is greater than the blade width W, which in turn is greater than blade thickness T. The blade portion 16 has two opposing faces 17 and 17′. While the blade portion 16 is depicted in the accompanying figures as planar, it is appreciated that a blade portion of an inventive callus rasp is readily contoured to illustratively include a concave surface and opposing convex surface. The rasp body 12 is formed of a nonporous and preferably autoclavable material. Materials from which the rasp body 12 is formed illustratively include stainless steel, anodized aluminum, glass, glazed ceramic, and brass. Preferably, the rasp body 12 is formed of surgical steel.

An abrasive surface 24 has an adhesive backing layer 26 for securement to the blade portion 16. An optional removable backing layer 28 protects the adhesive layer 26 prior to adhesion to the blade portion 16. A removable backing layer 28 is one conventional to the art with the exact composition dictated by the nature of the adhesive layer 26. A typical backing layer 28 is formed of paper having a low surface energy silicone or perfluoro polymer coating that promotes release from the adhesive layer 26. The abrasive surface 24 is formed by the adhesion of particulate of a predetermined coarseness to a substrate 30. The abrasive illustratively has a grit coarseness of between 5 and 800 where a higher grit number relates to a finer particulate. Tile substrate 30 preferably is able to bend to the contours of the blade portion 16 and is readily formed from paper, thin polymeric sheeting, and laminates therebetween. It is appreciated a second abrasive surface 74′ of like or varying grit are optionally applied simultaneously to a blade portion 16.

According to the present invention, a kit is provided inclusive of a rasp body 12 and at least one foil shown generally at 42. The foil 42 has abrasive surface 24, an adhesive layer 76 and a peelable release backing 28. The foil 42 is precut to conform with a surface of the blade portion 16 or alternatively is provided in a larger sheet to be custom cut to form an abrasive surface 24 for adherence to the blade portion 16. Preferably, a foil 42 is subjected to sterilization by such techniques as autoclave or gamma radiation sterilization and enclosure within a hermetically sealed package 46, shown in open form. As a result, a single user sterile abrasive surface is contacted with an individual precluding possible infection associated with previous individual debris on the rasp, as occurred with a prior art rasp.

According to the present invention, a user, such as a podiatrist, takes a foil 42 from a sterile package 46, if such package 46 is present, and removes the peelable backing 28 therefrom to expose the adhesive 26. The adhesive 26 is contacted with the blade portion 16 of a rasp body 12 that has been sterilized so as to adhere surface 24 to the blade portion of the rasp body, exposing the abrasive grit of the surface 24. This process of adhesion is optionally repeated with a second or further abrasive surface. It is appreciated that if the foil is oversized relative to the blade portion of the rasp body, that the foil is cut to a desired size and shape prior to removal of the backing and adhesive securement to the blade portion. With the abrasive surface now adhesively secured to the blade portion to form an inventive callus rasp, the callus rasp is then used to abrade the stratum corneum of an individual. After usage, tie abrasive surface 24 along with any second or further abrasive surface is removed and discarded while the rasp body is sterilized for repeated use.

The foregoing description is illustrative of particular embodiments of the invention, but is not meant to be a limitation upon the practice thereof. The following claims, including all equivalents thereof, are intended to define the scope of the invention.

1. A callus rasp comprising:
a rasp body having a handle portion and a body portion, said rasp body formed from a nonporous metal, glass, or glazed ceramic;
a sterile abrasive surface having particulate with a particulate grit size; and
a sterile peelably removable adhesive intermediate between the blade portion and the abrasive surface.

2. The rasp of claim 1 wherein said rasp body is a single piece of surgical steel.

3. The rasp of claim 1 wherein the abrasive surface is shaped to complement a face of the blade portion.

4. The rasp of claim 1 further comprising a second abrasive surface having particulate with a second abrasive surface particulate grit size exposed on the second abrasive surface, said second abrasive surface simultaneously secured to the blade portion.

5. The rasp of claim 4 wherein the particulate grit size is different than the second abrasive surface particulate grit size.

6. The rasp of claim I wherein the blade portion defines two planar opposing faces.

7. (canceled)

8. A callus rasp kit comprising:
a rasp body having a handle portion and a body portion, said rasp body formed from a nonporous metal, glass, or glazed ceramic;
at least one foil having an abrasive surface, an adhesive layer and a peelable backing layer in contact with said adhesive layer;
together with instructions for the removal of the peelable backing layer to secure the abrasive surface to the blade portion and operation as a callus rasp followed by abrasive surface removal and sterilization of said rasp body; and
a sealed package containing said foil in sterile form.

9. (canceled)

10. (canceled)

11. The kit of claim 8 wherein said foil is complementary to a face of the blade portion.

12. The kit of claim 8 wherein said foil is larger than the blade portion and instructions are provided to cut said foil to a desired shape prior to adhesion to the blade portion.

13. A process for sterile use of a callus rasp comprising:
removing a sterile foil from a sealed package, said foil having an abrasive surface, an adhesive layer and a peelable backing layer in contact with said adhesive layer;
peelably removing said peelable backing to expose said adhesive;
applying said adhesive to a body portion of a rasp body;
abrading a callus on an individual through contact with said abrasive surface; and
removing said adhesive from the body portion of said rasp body.

14. The process of claim 13 further comprising autoclave sterilizing said foil within said sealed package prior to the removing said sterile foil from said sealed package.

15. The process of claim 13 further comprising shaping said abrasive surface prior to the applying said adhesive to said body portion of said rasp body.

16. The process of claim 13 further comprising sterilizing said rasp body prior to applying said adhesive to said body portion of said rasp body.
